(19) United States
(12) Patent Application Publication
(10) Pub. No.: US 2020/0179254 A1
(43) Pub. Date: Jun. 11, 2020

(54) IMPROVED CONDITIONING HAIR TREATMENT PRODUCT WITH WASHOUT PROTECTION

(71) Applicant: Henkel AG & Co. KGaA, Duesseldorf (DE)

(72) Inventors: VOLKER SCHEUNEMANN, Lueneburg (DE); ERIK SCHULZE ZUR WIESCHE, Hamburg (DE); RENE KROHN, Norderstedt (DE)

(73) Assignee: Henkel AG & Co. KGaA, Duesseldorf (DE)

(21) Appl. No.: 16/337,295

(22) PCT Filed: Jul. 13, 2017
(86) PCT No.: PCT/EP2017/067689

§ 371 Date: Mar. 27, 2019

(30) Foreign Application Priority Data
Sep. 30, 2016 (DE) 10 2016 219 006.7

(51) Int. Cl. A61K 8/49; A61K 8/41; A61K 8/27; A61K 8/26; A61K 8/19; A61K 8/36; A61K 8/73; A61K 8/365; A61K 8/362; A61Q 5/12; A61Q 5/00

(52) U.S. Cl. CPC A61K 8/4913; A61K 8/416; A61K 8/27; A61K 8/26; A61K 8/19; A61Q 5/006; A61K 8/731; A61K 8/737; A61K 8/365; A61K 8/362; A61Q 5/12; A61K 8/36

(57) ABSTRACT

Hair treatment products comprising, in relation to its weight, from about 0.001 to about 10% by weight of at least one amine salt, of a carboxylic acid, at least one bivalent or trivalent metal salt and at least one organic acid, which lead to improved structural reinforcement of keratin fibres and improved hair care, and which reduce or prevent colour washout in dyed hair.

### CROSS-REFERENCE TO RELATED APPLICATION

This application is a U.S. National-Stage entry under 35 U.S.C. § 371 based on International Application No. PCT/EP2017/067689, filed Jul. 13, 2017, which was published under PCT Article 21(2) and which claims priority to German Application No. 10 2016 219 006.7, filed Sep. 30, 2016, which are all hereby incorporated in their entirety by reference.

### TECHNICAL FIELD

The present disclosure relates to hair treatment agents, in particular shampoos and what are referred to as conditioners, having an active ingredient combination for the gentle and effective care of hair.

### BACKGROUND

The importance of care products with the longest-lasting possible effect is increasing not least due to the high amount of strain on hair caused, for example, by coloring or permanents, by cleaning hair with shampoos, and by environmental pressures. Care products of this type influence the natural structure and properties of hair. For example, the wet and dry combability, the hold and the volume of hair can be optimized following care treatments of this kind, or hair can be protected from increased splitting.

It has long been customary, therefore, to subject hair to a special aftertreatment. In this process, the hair is treated with special active ingredients, for example quaternary ammonium salts or special polymers, usually in the form of a rinse. As a result of this treatment, depending on the formulation, the combability, the hold, and the volume of the hair are improved, and the amount of splitting is reduced.

Multi-functional cosmetic products are also known in the prior art. These include in particular what are referred to as “2 in 1” shampoos, which not only clean hair but also condition it. Products of this type are held in high esteem by consumers because, due to their product performance, they eradicate the need for at least one process step, for example conditioning with a conventional hair conditioner.

Similarly, products for modifying natural hair color play a prominent role in hair cosmetics. A distinction is made between permanent, semi-permanent, or temporary coloring systems based on chemical and/or natural dyes. Hair colors produced artificially by permanent, semi-permanent or temporary coloring systems are however disadvantageous in that they can change undesirably, for example during or after hair cleaning.

An “undesirable change” is understood to mean fading or bleeding and loss of the color brilliance of the hair shade achieved by the particular coloring. Environmental impacts and/or the effects of sunlight can further intensify these changes.

There continues to be a need to provide active ingredients or active ingredient combinations for hair treatment agents which have good nourishing properties and also strengthen the adhesion of dyes to hair fibers and thus maintain the fastness of the artificially produced hair color, and in this respect to develop hair treatment agents.

It has been observed, however, that water hardness can sometimes negatively influence the nourishing properties of hair treatment agents, and therefore the nourishing performance of the same product may be either too low or too high depending on water quality.

Nourishing performance that is too high or “over-nourishment” (occurrence of what is known as a build-up effect) of the hair is understood to mean in particular a greasy hair feel, lack of hair volume, and/or a lank, unkempt appearance of the hair, whereas damaged hair (broken and dull hair that is prone to splitting) is an indicator of too little nourishing performance.

### BRIEF SUMMARY

This disclosure provides a hair treatment agent including, based on its weight,
a) from about 0.001 to about 10 wt. % of at least one amine salt of a carboxylic acid;
b) at least one bivalent or trivalent metal salt;
c) at least one organic acid,

with the proviso that the agent comprises, based on its weight, from about 0.01 to about 10 wt. % of at least one amine salt from the group

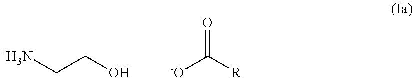

in which R represents -Ph or —(CH₂)nCH3, where n=4, 5, 6, 7, 8, 9 or 10,

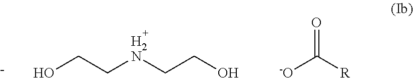

in which R represents -Ph or —(CH₂)nCH3, where n=4, 5, 6, 7, 8, 9 or 10,

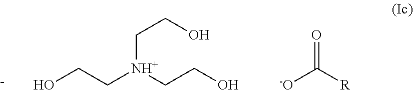

in which R represents -Ph or —(CH₂)nCH₃, where n=4, 5, 6, 7, 8, 9 or 10,

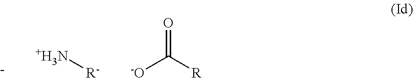

in which R and R′ represent, independently of one another, -Ph or —(CH₂)nCH3, where n=4, 5, 6, 7, 8, 9 or 10.

This disclosure also provides a method for treating hair wherein the aforementioned agent is applied to dry or wet hair, is left there for a period of from about 10 to about 300 seconds, and is rinsed out thereafter. This disclosure further provides a method for treating hair wherein the aforementioned agent is applied to dry or wet hair and is left there until the next time the hair is washed.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The object of the present application was therefore to provide skin-compatible, nourishing hair treatment agents which have excellent foaming properties, can be rinsed out well with water and additionally reduce or prevent washout of color from colored hair.

The nourishing hair treatment agents should have a uniform conditioning performance irrespective of water quality and in particular should not weigh down fine hair and/or damaged hair, but should strengthen the structure of the hair and thus protect it from splitting and breaking and should improve combability and the feel of the hair. In addition, said agents should have a germicidal effect.

It has now been found that a combination of certain ingredients has a particularly positive effect on colored hair and the hair follicles treated with said ingredients.

A first subject of the present disclosure is hair treatment agents containing, based on their weight,
a) from about 0.001 to about 10 wt. % of at least one amine salt of a carboxylic acid;
b) at least one bivalent or trivalent metal salt;
c) at least one organic acid.

Hair treatment agents within the meaning of the present disclosure are, for example, hair shampoos, hair conditioners, conditioning shampoos, hair sprays, hair rinses, hair masques, hair packs, hair tonics, permanent wave fixing solutions, hair coloring shampoos, hair dyes, hair setting lotions, hair setting products, hair styling preparations, blow-dry wave lotions, styling mousses, hair gels, hair waxes, or combinations thereof. In view of the fact that men in particular are often reluctant to use multiple different agents and/or multiple application steps, agents that men use already are preferred. Preferred agents are therefore shampoos, conditioning agents, or hair tonics.

The hair treatment agents contain, as a first essential ingredient, from about 0.001 to about 10 wt. % of at least one amine salt of a carboxylic acid.

Amine salts of carboxylic acids are formed from a (primary, secondary or tertiary) amine together with a carboxylic acid from the —COOH group thereof. In the process, the acidic H of the acid adds a proton to the amine in order to form an ammonium. Suitable amines are, for example, methylamine, dimethylamine, trimethylamine, ethylamine, diethylamine, triethylamine, n-propylamine, di-n-propylamine, tri-n-propylamine, isopropylamine, diisopropylamine, triisopropylamine, phenylamine (aniline), diphenylamine, triphenylamine, monoethanolamine, diethanolamine, triethanolamine, ethylenediamine, propylenediamine, the phenylenediamines 1,2-diaminobenzene, 1,3-diaminobenzene and 1,4-diaminobenzene, piperidine, morpholine, 4,4-dimethyloxazolidine, etc.

Typical representatives of aliphatic mono and dicarboxylic acids are, for example, acetic acid, propionic acid, oxalic acid, and 1,3-propanedioic acid, and aromatic carboxylic acids, such as benzoic acid. Other organic acids are, for example, hydroxycarboxylic acids such as glycolic acid, citric acid, tartaric acid, malic acid, and lactic acid. Unsaturated mono or dicarboxylic acids such as fumaric acid, or α-ketocarboxylic acids such as pyruvic acid (2-oxopropionic acid) can also be used as amine salts as contemplated herein.

Examples of amine salts of carboxylic acids that are preferably to be used as contemplated herein are methylammonium formate, dimethylammonium formate, trimethylammonium formate, ethylammonium formate, diethylammonium formate, triethylammonium formate, n-propylammonium formate, di-n-propylammonium formate, tri-n-propylammonium formate, isopropylammonium formate, diisopropylammonium formate, triisopropylammonium formate, phenylammonium formate, diphenylammonium formate, triphenylammonium formate, monoethanol ammonium formate, diethanol ammonium formate, triethanol ammonium formate, ethylene ammonium formate, propylene ammonium formate, piperidinium formate, morpholinium formate, 4,4-dimethyloxazolidine formate, methylammonium acetate, dimethylammonium acetate, trimethylammonium acetate, ethylammonium acetate, diethylammonium acetate, triethylammonium acetate, n-propylammonium acetate, di-n-propylammonium acetate, tri-n-propylammonium acetate, isopropylammonium acetate, diisopropylammonium acetate, triisopropylammonium acetate, phenylammonium acetate, diphenylammonium acetate, triphenylammonium acetate, monoethanol ammonium acetate, diethanol ammonium acetate, triethanol ammonium acetate, ethylene ammonium acetate, propylene ammonium acetate, piperidinium acetate, morpholinium acetate, 4,4-dimethyloxazolidine acetate, methylammonium propionate, dimethylammonium propionate, trimethylammonium propionate, ethylammonium propionate, diethylammonium propionate, triethylammonium propionate, n-propylammonium propionate, di-n-propylammonium propionate, tri-n-propylammonium propionate, isopropylammonium propionate, diisopropylammonium propionate, triisopropylammonium propionate, phenylammonium propionate, diphenylammonium propionate, triphenylammonium propionate, monoethanol ammonium propionate, diethanol ammonium propionate, triethanol ammonium propionate, ethylene ammonium propionate, propylene ammonium propionate, piperidinium propionate, morpholinium propionate, 4,4-dimethyloxazolidine propionate, methylammonium butyrate, dimethylammonium butyrate, trimethylammonium butyrate, ethylammonium butyrate, diethylammonium butyrate, triethylammonium butyrate, n-propylammonium butyrate, di-n-propylammonium butyrate, tri-n-propylammonium butyrate, isopropylammonium butyrate, diisopropylammonium butyrate, triisopropylammonium butyrate, phenylammonium butyrate, diphenylammonium butyrate, triphenylammonium butyrate, monoethanol ammonium butyrate, diethanol ammonium butyrate, triethanol ammonium butyrate, ethylene ammonium butyrate, propylene ammonium butyrate, piperidinium butyrate, morpholinium butyrate, 4,4-dimethyloxazolidine butyrate, methylammonium pentanoate, dimethylammonium pentanoate, trimethylammonium pentanoate, ethylammonium pentanoate, diethylammonium pentanoate, triethylammonium pentanoate, n-propylammonium pentanoate, di-n-propylammonium pentanoate, tri-n-propylammonium pentanoate, isopropylammonium pentanoate, diisopropylammonium pentanoate, triisopropylammonium pentanoate, phenylammonium pentanoate, diphenylammonium pentanoate, triphenylammonium pentanoate, monoethanol ammonium pentanoate, diethanol ammonium pentanoate, triethanol ammonium pentanoate, ethylene ammonium pentanoate, propylene ammonium pentanoate, piperidinium pentanoate, morpholinium pentanoate, 4,4-dimethyloxazolidine pentanoate, methylammonium hexanoate, dimethylammonium hexanoate, trimethylammonium hexanoate, ethylammonium hexanoate, diethylammonium hexanoate, triethylammonium hexanoate, n-propylammonium hexanoate, di-n-propylammonium hexanoate, tri-n-propylammonium hexanoate, isopropylammonium hexanoate, diisopropyl ammonium hexanoate, triisopropylammonium hexanoate, phenylammonium hexanoate, diphenylammonium hexanoate, triphenylammonium hexanoate, monoethanol ammonium hexanoate, diethanol ammonium hexanoate, triethanol ammonium hexanoate, ethylene ammonium hexanoate, propylene ammonium hexanoate, piperidinium hexanoate, morpholinium hexanoate, 4,4-dimethyloxazolidine hexanoate, methylammonium heptanoate, dimethylammonium heptanoate, trimethylammonium heptanoate, ethylammonium heptanoate, diethylammonium heptanoate, triethylammonium heptanoate, n-propylammonium heptanoate, di-n-propylammonium heptanoate, tri-n-propylammonium heptanoate, isopropylammonium heptanoate, diisopropylammonium heptanoate, triisopropylammonium heptanoate, phenylammonium heptanoate, diphenylammonium heptanoate, triphenylammonium heptanoate, monoethanol ammonium heptanoate, diethanol ammonium heptanoate, triethanol ammonium heptanoate, ethylene ammonium heptanoate, propylene ammonium heptanoate, piperidinium heptanoate, morpholinium heptanoate, 4,4-dimethyloxazolidine heptanoate, methylammonium octanoate, dimethylammonium octanoate, trimethylammonium octanoate, ethylammonium octanoate, diethylammonium octanoate, triethylammonium octanoate, n-propylammonium octanoate, di-n-propylammonium octanoate, tri-n-propylammonium octanoate, isopropylammonium octanoate, diisopropylammonium octanoate, triisopropylammonium octanoate, phenylammonium octanoate, diphenylammonium octanoate, triphenylammonium octanoate, monoethanol ammonium octanoate, diethanol ammonium octanoate, triethanol ammonium octanoate, ethylene ammonium octanoate, propylene ammonium octanoate, piperidinium octanoate, morpholinium octanoate, 4,4-dimethyloxazolidine octanoate, methylammonium nonanoate, dimethylammonium nonanoate, trimethylammonium nonanoate, ethylammonium nonanoate, diethylammonium nonanoate, triethylammonium nonanoate, n-propylammonium nonanoate, di-n-propylammonium nonanoate, tri-n-propylammonium nonanoate, isopropylammonium nonanoate, diisopropylammonium nonanoate, triisopropylammonium nonanoate, phenylammonium nonanoate, diphenylammonium nonanoate, triphenylammonium nonanoate, monoethanol ammonium nonanoate, diethanol ammonium nonanoate, triethanol ammonium nonanoate, ethylene ammonium nonanoate, propylene ammonium nonanoate, piperidinium nonanoate, morpholinium nonanoate, 4,4-dimethyloxazolidine nonanoate, methylammonium decanoate, dimethylammonium decanoate, trimethylammonium decanoate, ethylammonium decanoate, diethylammonium decanoate, triethylammonium decanoate, n-propylammonium decanoate, di-n-propylammonium decanoate, tri-n-propylammonium decanoate, isopropylammonium decanoate, diisopropylammonium decanoate, triisopropylammonium decanoate, phenylammonium decanoate, diphenylammonium decanoate, triphenylammonium decanoate, monoethanol ammonium decanoate, diethanol ammonium decanoate, triethanol ammonium decanoate, ethylene ammonium decanoate, propylene ammonium decanoate, piperidinium decanoate, morpholinium decanoate, 4,4-dimethyloxazolidine decanoate, methylammonium undecanoate, dimethylammonium undecanoate, trimethylammonium undecanoate, ethylammonium undecanoate, diethylammonium undecanoate, triethylammonium undecanoate, n-propylammonium undecanoate, di-n-propylammonium undecanoate, tri-n-propylammonium undecanoate, isopropylammonium undecanoate, diisopropylammonium undecanoate, triisopropylammonium undecanoate, phenylammonium undecanoate, diphenylammonium undecanoate, triphenylammonium undecanoate, monoethanol ammonium undecanoate, diethanol ammonium undecanoate, triethanol ammonium undecanoate, ethylene ammonium undecanoate, propylene ammonium undecanoate, piperidinium undecanoate, morpholinium undecanoate, 4,4-dimethyloxazolidine undecanoate, methylammonium dodecanoate, dimethylammonium dodecanoate, trimethylammonium dodecanoate, ethylammonium dodecanoate, diethylammonium dodecanoate, triethylammonium dodecanoate, n-propylammonium dodecanoate, di-n-propylammonium dodecanoate, tri-n-propylammonium dodecanoate, isopropylammonium dodecanoate, diisopropylammonium dodecanoate, triisopropylammonium dodecanoate, phenylammonium dodecanoate, diphenylammonium dodecanoate, triphenylammonium dodecanoate, monoethanol ammonium dodecanoate, diethanol ammonium dodecanoate, triethanol ammonium dodecanoate, ethylene ammonium dodecanoate, propylene ammonium dodecanoate, piperidinium dodecanoate, morpholinium dodecanoate, 4,4-dimethyloxazolidine dodecanoate, methylammonium oxalate, dimethylammonium oxalate, trimethylammonium oxalate, ethylammonium oxalate, diethylammonium oxalate, triethylammonium oxalate, n-propylammonium oxalate, di-n-propylammonium oxalate, tri-n-propylammonium oxalate, isopropylammonium oxalate, diisopropylammonium oxalate, triisopropylammonium oxalate, phenylammonium oxalate, diphenylammonium oxalate, triphenylammonium oxalate, monoethanol ammonium oxalate, diethanol ammonium oxalate, triethanol ammonium oxalate, ethylene ammonium oxalate, propylene ammonium oxalate, piperidinium oxalate, morpholinium oxalate, 4,4-dimethyloxazolidine oxalate, methylammonium benzoate, dimethylammonium benzoate, trimethylammonium benzoate, ethylammonium benzoate, diethylammonium benzoate, triethylammonium benzoate, n-propylammonium benzoate, di-n-propylammonium benzoate, tri-n-propylammonium benzoate, isopropylammonium benzoate, diisopropylammonium benzoate, triisopropylammonium benzoate, phenylammonium benzoate, diphenylammonium benzoate, triphenylammonium benzoate, monoethanol ammonium benzoate, diethanol ammonium benzoate, triethanol ammonium benzoate, ethylene ammonium benzoate, propylene ammonium benzoate, piperidinium benzoate, morpholinium benzoate, 4,4-dimethyloxazolidine benzoate, methylammonium oxalate, dimethylammonium oxalate, trimethylammonium oxalate, ethylammonium oxalate, diethylammonium oxalate, triethylammonium oxalate, n-propylammonium oxalate, di-n-propylammonium oxalate, tri-n-propylammonium oxalate, isopropylammonium oxalate, diisopropylammonium oxalate, triisopropylammonium oxalate, phenylammonium oxalate, diphenylammonium oxalate, triphenylammonium oxalate, monoethanol ammonium oxalate, diethanol ammonium oxalate, triethanol ammonium oxalate, ethylene ammonium oxalate, propylene ammonium oxalate, piperidinium oxalate, morpholinium oxalate, 4,4-dimethyloxazolidine oxalate, methylammonium glycolate, dimethylammonium glycolate, trimethylammonium glycolate, ethylammonium glycolate, diethylammonium glycolate, triethylammonium glycolate, n-propylammonium glycolate, di-n-propylammonium glycolate, tri-n-propylammonium glycolate, isopropylammonium glycolate, diisopropylammonium glycolate, triisopropylammonium glycolate, phenylammonium glycolate, diphenylammonium glycolate, triphenylammonium glycolate, monoethanol ammonium glycolate, diethanol ammonium glycolate, triethanol ammonium glycolate, ethylene ammonium glycolate, propylene ammonium glycolate, piperidinium glycolate, morpholinium glycolate, 4,4-dimethyloxazolidine glycolate, methylammonium citrate, dimethylammonium citrate, trimethylammonium citrate, ethylammonium citrate, diethylammonium citrate, triethylammonium citrate, n-propylammonium citrate, di-n-propylammonium citrate, tri-n-propylammonium citrate, isopropylammonium citrate, diisopropylammonium citrate, triisopropylammonium citrate, phenylammonium citrate, diphenylammonium citrate, triphenylammonium citrate, monoethanol ammonium citrate, diethanol ammonium citrate, triethanol ammonium citrate, ethylene ammonium citrate, propylene ammonium citrate, piperidinium citrate, morpholinium citrate, 4,4-dimethyloxazolidine citrate, methylammonium tartrate, dimethylammonium tartrate, trimethylammonium tartrate, ethylammonium tartrate, diethylammonium tartrate, triethylammonium tartrate, n-propylammonium tartrate, di-n-propylammonium tartrate, tri-n-propylammonium tartrate, isopropylammonium tartrate, diisopropylammonium tartrate, triisopropylammonium tartrate, phenylammonium tartrate, diphenylammonium tartrate, triphenylammonium tartrate, monoethanol ammonium tartrate, diethanol ammonium tartrate, triethanol ammonium tartrate, ethylene ammonium tartrate, propylene ammonium tartrate, piperidinium tartrate, morpholinium tartrate, 4,4-dimethyloxazolidine tartrate, methylammonium malate, dimethylammonium malate, trimethylammonium malate, ethylammonium malate, diethylammonium malate, triethylammonium malate, n-propylammonium malate, di-n-propylammonium malate, tri-n-propylammonium malate, isopropylammonium malate, diisopropylammonium malate, triisopropylammonium malate, phenylammonium malate, diphenylammonium malate, triphenylammonium malate, monoethanol ammonium malate, diethanol ammonium malate, triethanol ammonium malate, ethylene ammonium malate, propylene ammonium malate, piperidinium malate, morpholinium malate, 4,4-dimethyloxazolidine malate, methylammonium lactate, dimethylammonium lactate, trimethylammonium lactate, ethylammonium lactate, diethylammonium lactate, triethylammonium lactate, n-propylammonium lactate, di-n-propylammonium lactate, tri-n-propylammonium lactate, isopropylammonium lactate, diisopropylammonium lactate, triisopropylammonium lactate, phenylammonium lactate, diphenylammonium lactate, triphenylammonium lactate, monoethanol ammonium lactate, diethanol ammonium lactate, triethanol ammonium lactate, ethylene ammonium lactate, propylene ammonium lactate, piperidinium lactate, morpholinium lactate, 4,4-dimethyloxazolidine lactate, methylammonium fumarate, dimethylammonium fumarate, trimethylammonium fumarate, ethylammonium fumarate, diethylammonium fumarate, triethylammonium fumarate, n-propylammonium fumarate, di-n-propylammonium fumarate, tri-n-propylammonium fumarate, isopropylammonium fumarate, diisopropylammonium fumarate, triisopropylammonium fumarate, phenylammonium fumarate, diphenylammonium fumarate, triphenylammonium fumarate, monoethanol ammonium fumarate, diethanol ammonium fumarate, triethanol ammonium fumarate, ethylene ammonium fumarate, propylene ammonium fumarate, piperidinium fumarate, morpholinium fumarate, 4,4-dimethyloxazolidine fumarate, methylammonium pyruvate, dimethylammonium pyruvate, trimethylammonium pyruvate, ethylammonium pyruvate, diethylammonium pyruvate, triethylammonium pyruvate, n-propylammonium pyruvate, di-n-propylammonium pyruvate, tri-n-propylammonium pyruvate, isopropylammonium pyruvate, diisopropylammonium pyruvate, triisopropylammonium pyruvate, phenylammonium pyruvate, diphenylammonium pyruvate, triphenylammonium pyruvate, monoethanol ammonium pyruvate, diethanol ammonium pyruvate, triethanol ammonium pyruvate, ethylene ammonium pyruvate, propylene ammonium pyruvate, piperidinium pyruvate, morpholinium pyruvate, 4,4-dimethyloxazolidine pyruvate.

Particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of amine salt(s) of carboxylic acid(s) of formula (I)

in which
R1, R2 and R3 are selected, independently of one another, from —H, —CH₃, —CH₂CH₃, —CH₂CH₂OH,

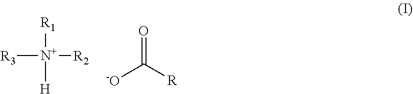

R represents an optionally substituted saturated or unsaturated, linear, branched or cyclic, aliphatic or aromatic hydrocarbon functional group having at least 2 C atoms.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of at least one amine salt from the group

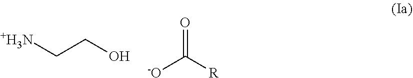

in which R represents -Ph or —(CH₂)nCH3, where n=4, 5, 6, 7, 8, 9 or 10, in particular 5 or 7,

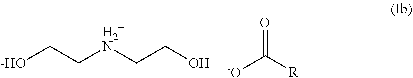

in which R represents -Ph or —(CH₂)nCH3, where n=4, 5, 6, 7, 8, 9 or 10, in particular 5 or 7,

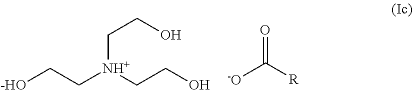

in which R represents -Ph or —(CH₂)nCH₃, where n=4, 5, 6, 7, 8, 9 or 10, in particular 5 or 7,

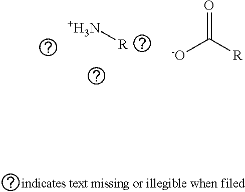

in which R and R′ represent, independently of one another, -Ph or —(CH₂)nCH3, where n=4, 5, 6, 7, 8, 9 or 10, in particular 5 or 7.

The hair treatment agents contain at least one divalent or trivalent metal salt.

To achieve an optimum effect, it is advantageous for the metal salts to be present in dissolved form in the agents as contemplated herein. In a preferred embodiment, the hair cleaning and care agents as contemplated herein therefore contain water-soluble bivalent or trivalent metal salts. “Water-soluble” is understood here to mean that, at about 20° C., at least about 1 g of the salt in question can dissolve fully in about 1 L of water.

Suitable divalent or trivalent metal salts may be selected from divalent or trivalent organic and/or inorganic salts.

Particularly suitable cations within these salts may preferably be selected from alkaline-earth metal cations and from copper, zinc, iron(II), iron(III), lanthanum(III) and/or aluminum cations.

Alkaline-earth metal cations, and particularly preferably calcium and magnesium cations, are very particularly preferred. Particularly suitable organic anions within these salts may preferably be selected from acetate, lactate, succinate, citrate, tartrate, malate, maleate, oxalate, and/or glycolate ions. Acetate, lactate, and/or citrate salts having the aforementioned cations are very particularly preferred.

Particularly preferred organic salts are calcium lactate, calcium citrate, calcium acetate, magnesium lactate, magnesium citrate, and/or magnesium acetate.

Particularly suitable inorganic anions within these salts may be selected from halide, sulfate, phosphate, and/or carbonate ions. Sulfate and/or halide ions such as chloride and bromide ions are very particularly preferred.

Particularly preferred inorganic salts are calcium chloride, calcium sulfate, magnesium chloride, and/or magnesium sulfate.

The weight proportion of the at least one bivalent or trivalent metal salt in terms of the total weight of the hair treatment agents as contemplated herein is preferably from about 0.01 to about 10 wt. %, more preferably from about 0.1 to about 7.5 wt. %, even more preferably from about 0.2 to about 5 wt. %, and in particular from about 0.3 to about 3 wt. %.

Hair treatment agents which are preferred as contemplated herein contain, based on their weight, from about 0.01 to about 10 wt. %, preferably from about 0.1 to about 7.5 wt. %, more preferably from about 0.2 to about 5 wt. %, and in particular from about 0.3 to about 3 wt. %, of at least one bivalent or trivalent metal salt from the group of organic or inorganic copper, zinc, iron(II), calcium, magnesium, iron(III), and/or aluminum salts.

Water-soluble salts are particularly preferred in this embodiment. Calcium lactate, calcium citrate, calcium acetate, magnesium lactate, magnesium citrate, magnesium acetate, calcium halides, calcium hydroxide, magnesium halides, and/or magnesium hydroxide are very particularly preferred in this embodiment.

The hair treatment agents contain at least one organic acid.

Typical representatives of organic acids are aliphatic mono and dicarboxylic acids, such as acetic acid, propionic acid, oxalic acid, and 1,3-propanedioic acid, and aromatic carboxylic acids, such as benzoic acid. Other organic acids are, for example, hydroxycarboxylic acids such as glycolic acid, citric acid, tartaric acid, malic acid, and lactic acid. Unsaturated mono or dicarboxylic acids such as fumaric acid, or α-ketocarboxylic acids such as pyruvic acid (2-oxopropionic acid) are also part of the present disclosure.

Hair treatment agents which are preferred as contemplated herein contain, based on their weight, from about 0.001 to about 10 wt. %, preferably from about 0.01 to about 5 wt. %, more preferably from about 0.05 to about 3 wt. %, and in particular from about 0.1 to about 2 wt. %, of organic acid(s).

Particularly preferred hair treatment agents contain, based on their weight, from about 0.001 to about 10 wt. %, preferably from about 0.01 to about 5 wt. %, more preferably from about 0.05 to about 3 wt. %, and in particular from about 0.1 to about 2 wt. %, of organic acid(s) from the group of formic acid, acetic acid, propionic acid, butyric acid, isobutyric acid, valeric acid, isovaleric acid, pivalic acid, oxalic acid, malonic acid, succinic acid, glutaric acid, glyceric acid, glyoxylic acid, adipic acid, pimelic acid, suberic acid, azelaic acid, sebacic acid, propiolic acid, crotonic acid, isocrotonic acid, elaidic acid, maleic acid, fumaric acid, muconic acid, citraconic acid, mesaconic acid, camphoric acid, benzoic acid, o,m,p-phthalic acid, naphthoic acid, toluic acid, hydratropic acid, atropic acid, cinnamic acid, isonicotinic acid, nicotinic acid, bicarbaminic acid, 4,4′-dicyano-6,6′-binicotinic acid, 8-carbamoyloctanoic acid, 1,2,4-pentanetricarboxylic acid, 2-pyrrole carboxylic acid, 1,2,4,6,7-napthalene pentaacetic acid, malonaldehydic acid, 4-hydroxyphthalamidic acid, 1-pyrazole carboxylic acid, gallic acid or propane tricarboxylic acid, glycolic acid, lactic acid, malic acid, tartaric acid, or citric acid.

Some representatives are particularly preferred from among the aforementioned acids, because they additionally impart a pleasant shine to hair treated with the agents as contemplated herein. Extremely preferred hair treatment agents contain, based on their weight, from about 0.001 to about 10 wt. %, preferably from about 0.01 to about 5 wt. %, more preferably from about 0.05 to about 3 wt. %, and in particular from about 0.1 to about 2 wt. %, of organic acid(s) from the group of formic acid, oxalic acid, maleic acid, lactic acid, tartaric acid, succinic acid, or citric acid.

The agents as contemplated herein may contain amino-functional silicones to further increase the conditioning effects. Preferred agents as contemplated herein contain from about 0.001 to about 50 wt. % of at least one amino-functional silicone.

Particularly preferred agents as contemplated herein contain at least one amino-functional silicone of formula (Si-IIa)

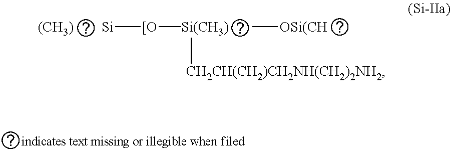

in which m and n are numbers whose sum (m+n) is between about 1 and about 2000, preferably between about 50 and about 150, n preferably assuming values of from 0 to about 1999 and in particular from about 49 to about 149 and m preferably assuming values of from about 1 to about 2000, in particular from about 1 to about 10.

These silicones are referred to as trimethylsilylamodimethicone according to the INCI declaration.

Particularly preferred as well are agents as contemplated herein that contain an amino-functional silicone of formula (Si-IIb)

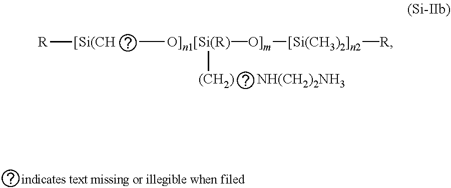

in which R represents —OH, —O—CH₃, or a —CH₃ group and m, n1 and n2 are numbers whose sum (m+n1+n2) is between about 1 and about 2000, preferably between about 50 and about 150, the sum (n1+n2) preferably assuming values of from 0 to about 1999 and in particular from about 49 to about 149 and m preferably assuming values of from about 1 to about 2000, in particular from about 1 to about 10.

These silicones are referred to as amodimethicone according to the INCI declaration.

Irrespective of which amino-functional silicones are used, agents as contemplated herein are preferred that contain an amino-functional silicone of which the amine value is above about 0.25 meq/g, preferably above about 0.3 meq/g, and in particular above about 0.4 meq/g. The amine value here represents the milliequivalents of amine per gram of the amino-functional silicone. Said value can be determined by titration and may also be given in the unit mg KOH/g.

Hair treatment agents which are preferred as contemplated herein contain, based on their weight, from about 0.01 to about 20 wt. %, preferably from about 0.05 to about 10 wt. %, more preferably from about 0.1 to about 7.5 wt. %, and in particular from about 0.15 to about 5 wt. %, of amino-functional silicone(s).

The agents as contemplated herein particularly preferably contain amino-functional silicone(s) having terminal hydroxy group(s). Some special amino-functional silicone(s) having terminal hydroxy group(s) have been found to be particularly suitable in the agents as contemplated herein. These are described in the following.

Pretreatment agents that contain at least one silicone of formula (Si-V) have been found to be particularly effective in the method as contemplated herein with regard to the desired effects:

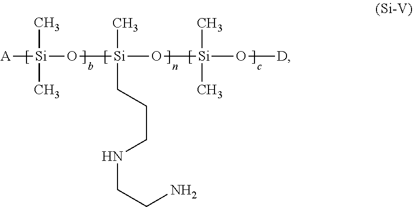

in which
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c represent integers between 0 and about 1000,
   with the proviso that
      n>0 and b+c>0
      at least one of the conditions A=—OH or D=—H is met.

Hair treatment agents as contemplated herein are therefore preferred as contemplated herein that contain, based on their weight, from about 0.01 to about 20 wt. %, preferably from about 0.01 to about 20 wt. %, more preferably from about 0.1 to about 10 wt. %, even more preferably from about 0.5 to about 7.5 wt. %, and in particular from about 0.1 to about 5 wt. %, of at least one silicone of formula (Si-V):

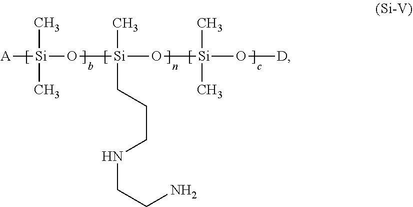

in which
A represents an —OH, —O—Si(CH₃)₃, —O—Si(CH₃)₂OH, or —O—Si(CH₃)₂OCH₃ group,
D represents an —H, —Si(CH₃)₃, —Si(CH₃)₂OH, or —Si(CH₃)₂OCH₃ group,
b, n and c represent integers between 0 and about 1000,
   with the proviso that
      n>0 and b+c>0
      at least one of the conditions A=—OH or D=—H is met.

In the above formula (Si-V), the individual siloxane units having the indices b, c and n are randomly distributed, i.e. they are not necessarily block copolymers.

Further particularly suitable silicones are 4-morpholinomethyl-substituted. Hair treatment agents as contemplated herein are particularly preferred that contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. %, of at least one 4-morpholinomethyl-substituted silicone which comprises structural units of formulas (Si-Via), (Si-VIb) and (Si-VIC)

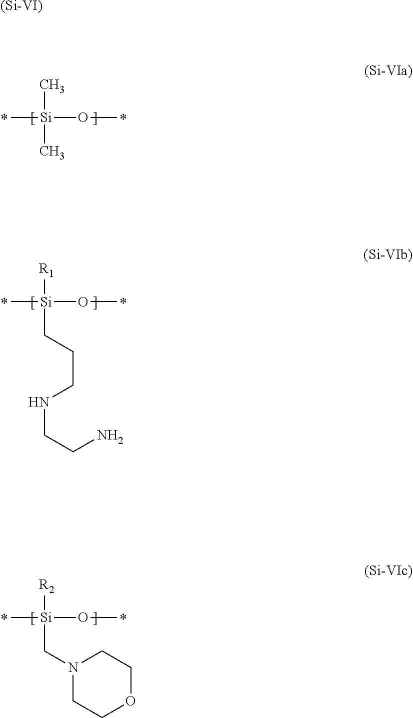

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃.

Particularly preferred hair treatment agents as contemplated herein contain, based on their weight, from about 0.001 to about 20 wt. %, preferably from about 0.01 to about 10 wt. %, particularly preferably from about 0.05 to about 7.5 wt. %, and in particular from about 0.5 to about 5 wt. %, of at least one 4-morpholinomethyl-substituted silicone of formula (Si-VI)

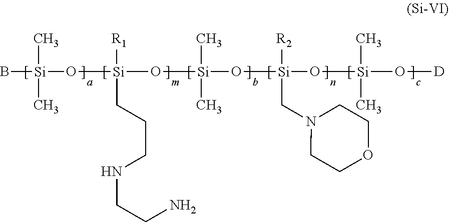

in which
R1 represents —CH₃, —OH, —OCH₃, —O—CH₂CH₃, —O—CH₂CH₂CH₃, or —O—CH(CH₃)₂;
R2 represents —CH₃, —OH, or —OCH₃;
B represents an —OH, —O—Si(CH₃)₃, —O—Si(CH₃)₂OH, or —O—Si(CH₃)₂OCH₃ group;
D represents an —H, —Si(CH₃)₃, —Si(CH₃)₂OH, or —Si(CH₃)₂OCH₃ group;
a, b and c represent, independently of one another, integers between 0 and about 1000,
with the proviso that a+b+c>0;
m and n represent, independently of one another, integers between about 1 and about 1000,
with the proviso that
   at least one of the conditions B=—OH or D=—H is met,
   the units a, b, c, m and n are distributed randomly or in blocks in the molecule.

Structural formula (Si-VI) is intended to indicate that the siloxane groups n and m do not necessarily have to be directly bonded to an end group B or D. Instead, in preferred formulas (Si-VI), a>0 or b>0 and, in particularly preferred formulas (Si-VI), a>0 and c>0; i.e., the terminal group B or D is preferably bound to a dimethylsiloxy group. In formula (Si-VI) as well, the siloxane units a, b, c, m and n are preferably distributed randomly.

The silicones represented by formula (Si-VI) and used as contemplated herein can be trimethylsilyl-terminated (D or B=—Si(CH₃)₃), but they may also be dimethylsilylhydroxy-terminated at both ends or dimethylsilylhydroxy-terminated at one end and dimethylsilylmethoxy-terminated at the other end. In the context of the present disclosure, silicones used particularly preferably are selected from silicones in which B=—O—Si(CH₃)₂OH and D=—Si(CH₃)₃ B=—O—Si(CH₃)₂OH and D=—Si(CH₃)₂OH B=—O—Si(CH₃)₂OH and D=—Si(CH₃)₂OCH₃ B=—O—Si(CH₃)₃ and D=—Si(CH₃)₂OH B=—O—Si(CH₃)₂OCH₃ and D=—Si(CH₃)₂OH.

These silicones lead to enormous improvements in the hair properties of hair treated with the agents as contemplated herein, and to greatly improved protection during oxidative treatment.

Irrespective of the type of amino-functional silicone(s) having terminal hydroxy group(s) used, the agents as contemplated herein contain the silicone(s) preferably in the form of an emulsion, particularly preferably in the form of a microemulsion.

It has been found that the effect of the silicones used in the agents as contemplated herein can be increased further still if certain nonionic components are also used in the agents. In addition, these nonionic components have positive effects on the storage stability of the agents. Nonionic components that are particularly suitable here are ethoxylates of decanol, undecanol, dodecanol, tridecanol, etc. Ethoxylated tridecanols which are particularly preferably incorporated in the agents as contemplated herein have been found to be particularly suitable. Agents which are particularly preferred as contemplated herein contain, based on their weight, from about 0.00001 to about 5 wt. %, preferably from about 0.0001 to about 3.5 wt. %, particularly preferably from about 0.001 to about 2 wt. %, more preferably from about 0.01 to about 1 wt. %, and in particular from about 0.1 to about 0.5 wt. %, of branched ethoxylated tridecanol (INCI name: trideceth-5) or α-iso-tridecyl-ω-hydroxy polyglycol ether (INCI name: trideceth-10) or mixtures thereof.

In addition to or instead of the amino-functional silicone(s), the agents as contemplated herein may contain further silicone(s) which is/are not amino-functional. The hair treatment agents as contemplated herein may contain surfactant(s). In cleaning compositions (shampoos), in particular anionic surfactants have been found to be suitable and, in conditioning compositions, cationic surfactants are ingredients that are often used; owing to their advantageous properties, amphoteric surfactants are particularly preferably used in both shampoos and conditioners.

The hair treatment agents as contemplated herein may contain at least one anionic surfactant.

Suitable anionic surfactants and emulsifiers for the compositions as contemplated herein include all anionic surface-active substances suitable for use on the human body. These are exemplified by a water-solubilizing, anionic group such as a carboxylate, sulfate, sulfonate or phosphate group and a lipophilic alkyl group having approximately 8 to 30 C atoms. In addition, glycol or polyglycol ether groups, ester, ether and amide groups, and hydroxyl groups, may additionally be contained in the molecule.

Preferred hair treatment agents contain, based on their weight, from about 0.5 to about 20 wt. %, preferably from about 0.75 to about 15 wt. %, more preferably from about 1 to about 12 wt. %, and in particular from about 2 to about 10 wt. %, of anionic surfactant(s).

Particularly preferred hair treatment agents as contemplated herein contain, based on their weight, from about 0.5 to about 20 wt. %, preferably from about 0.75 to about 15 wt. %, more preferably from about 1 to about 12 wt. %, and in particular from about 2 to about 10 wt. %, of alkyl (ether) sulfates of general formula R—(OCH₂—CH₂)ₙ—OSO₃X, in which R is a straight-chain or branched, saturated or unsaturated alkyl group having about 8 to about 24 C atoms, n is the number 0 or from about 1 to about 12, and X is an alkali, alkaline-earth, ammonium or alkanolamine ion.

The hair treatment agents as contemplated herein may contain at least one amphoteric surfactant and/or at least one nonionic surfactant.

Particularly preferred hair treatment agents as contemplated herein contain, based on their weight, from about 0.3 to about 20 wt. %, preferably from about 0.5 to about 8 wt. %, more preferably from about 0.75 to about 6 wt. %, and in particular from about 1 to about 5 wt. %, of amphoteric surfactant(s). Surfactants that have both a negatively-charged functional group and a positively-charged functional group are referred to as amphoteric surfactants or zwitterionic surfactants.

Particularly suitable zwitterionic surfactants are what are referred to as betaines, such as N-alkyl-N,N-dimethylammonium glycinates, for example cocoalkyl dimethylammonium glycinate, N-acylaminopropyl-N,N-dimethylammonium glycinates, for example cocoacylaminopropyl dimethylammonium glycinate, 2-alkyl-3-carboxymethyl-3-hydroxyethyl imidazolines each having about 8 to about 18 C atoms in the alkyl or acyl group, and cocoacylaminoethyl hydroxyethyl carboxymethyl glycinate. A preferred zwitterionic surfactant is the fatty acid amide derivative known by the INCI name cocamidopropyl betaine.

The hair treatment agents may contain nonionic surfactant(s).

Hair treatment agents which are preferred as contemplated herein contain, based on their weight, from about 0.3 to about 10 wt. %, preferably from about 0.5 to about 8 wt. %, more preferably from about 0.75 to about 6 wt. %, and in particular from about 1 to about 5 wt. %, of nonionic surfactant(s).

The hair treatment agents may contain cationic surfactant(s). As contemplated herein, it is possible to use cationic surfactants of the following types: quaternary ammonium compounds, esterquats, and amidoamines. Preferred quaternary ammonium compounds are ammonium halides, in particular chlorides and bromides, such as alkyl trimethylammonium chlorides, dialkyl dimethylammonium chlorides, and trialkyl methylammonium chlorides. The long alkyl chains of these surfactants preferably have about 10 to about 18 carbon atoms, such as in cetyl trimethylammonium chloride, stearyl trimethylammonium chloride, distearyl dimethylammonium chloride, lauryl dimethylammonium chloride, lauryl dimethylbenzylammonium chloride, and tricetyl methylammonium chloride. Further preferred cationic surfactants are the imidazolium compounds known by the INCI names quaternium-27 and quaternium-83.

Particularly preferred hair treatment agents as contemplated herein contain as the cationic care substance, based on their weight, from about 0.05 to about 7.5 wt. %, preferably from about 0.1 to about 5 wt. %, particularly preferably from about 0.2 to about 3.5 wt. %, and in particular from about 0.25 to about 2.5 wt. %, of cationic surfactant(s) from the group of quaternary ammonium compounds and/or esterquats and/or amidoamines.

Particularly preferred hair treatment agents as contemplated herein contain, based on the weight of the agent, from about 0.05 to about 20 wt. %, preferably from about 0.1 to about 10 wt. %, more preferably from about 0.25 to about 8 wt. %, and in particular from about 0.5 to about 7 wt. %, of cationic surfactant(s), and preferably from about 0.05 wt. % to about 20 wt. %, more preferably from about 0.1 to about 10 wt. %, even more preferably from about 0.25 to about 8 wt. %, and in particular from about 0.5 to about 7 wt. %, of behenyl trimethyl ammonium chloride.

The agents as contemplated herein may contain at least one cationic polymer.

Cationic polysaccharide polymers increase the nourishing performance of the hair treatment agents as contemplated herein (in particular the efficacy of the agents as contemplated herein with respect to hair breakage). Suitable cationic polysaccharide polymers may be selected from cationic cellulose compounds and/or from cationic guar derivatives.

Particularly preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of at least one polymer from the group of cationic cellulose polymers and/or cationic guar derivatives.

Cationic cellulose compounds within the meaning of the present disclosure are those that carry more than one permanent cationic charge in at least one side chain. Cellulose is constructed from beta-1,4-glycosidically linked D-glucopyranose units and forms unbranched, water-insoluble chains. A “side chain” of a cellulose defines chemical substituents which bind to the cellulose backbone and are not part of the native cellulose, since they have been introduced subsequently, for example by chemical synthesis.

Quaternized cellulose polymers originating from hydroxy(C₂-C₄)alkyl celluloses, particularly preferably from hydroxyethyl celluloses, are preferred.

Polymers of this kind are known to a person skilled in the art and are commercially available from various companies. The cationic cellulose derivatives known by the INCI names polyquaternium-4, polyquaternium-10, polyquaternium-24, polyquaternium-67, and/or polyquaternium-72 are particularly preferred. Polyquaternium-10, polyquaternium-24, and/or polyquaternium-67 are very particularly preferred, with polyquaternium-10 being particularly preferred.

Preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt, %, of at least one polymer from the group of polyquaternium-4, polyquaternium-10, polyquaternium-24, polyquaternium-67, and/or polyquaternium-72.

Particularly preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of polyquaternium-10.

Suitable cationic guar derivatives within the meaning of the present disclosure are cationic hydroxyalkyl guar derivatives, preferably cationic hydroxyethyl trimethylammonium guar and/or cationic hydroxypropyl trimethylammonium guar having average molecular weights between about 100,000 and about 2,000,000 daltons. The cationic guar polymers known by the INCI name guar hydroxypropyltrimonium chloride having a molecular weight (weight average) between about 200,000 and about 1,600,000 daltons are particularly preferred. The cationic charge density of these guar polymers is preferably at least about 0.4 meq/g, preferably at least about 0.5 meq/g, and in particular at least about 0.6 meq/g. Their nitrogen content is preferably in a range of from about 1.1 to about 1.8 wt. % (based on their total weight).

Cationic guar derivatives known by the INCI name guar hydroxypropyltrimonium chloride are known to a person skilled in the art and are obtainable for example under the trade names Cosmedia® Guar, N-Hance® and/or Jaguar® from various providers.

Particularly preferred hair treatment agents as contemplated herein contain as cationic polysaccharide polymer(s), based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of guar hydroxypropyltrimonium chloride.

In summary, hair treatment agents which are preferred as contemplated herein contain, based on the weight of the agent, from about 0.01 to about 3 wt. %, preferably from about 0.05 to about 2 wt. %, more preferably from about 0.1 to about 1.5 wt. %, and in particular from about 0.15 to about 0.8 wt. %, of cationic polymer(s), and preferably from about 0.01 wt. % to about 3 wt. %, more preferably from about 0.05 to about 2 wt. %, even more preferably from about 0.1 to about 1.5 wt. %, and in particular from about 0.15 to about 0.8 wt. %, of at least one polymer from the group of cationic cellulose polymers and/or cationic guar derivatives.

It has been found that alpha-substituted aldehydes can further enhance the effect of the agents as contemplated herein. In particular, significant increases in performance are observed with regard to the inner structural strengthening of keratin fibers and the prevention of washout.

Hair treatment agents which are preferred as contemplated herein additionally contain, based on the weight of the agent, from about 0.001 to about 20 wt. % of at least one alpha-substituted aldehyde.

Preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of alpha-substituted aldehyde(s) of formula (I)

Y—CH(X)—CHO (I),

in which
X represents —OH, —Cl, —Br, —I, —O—(CH₂)ₙ—CH₃ where n=1, 2, 3 or 4, or —O—(CH₂)ₘ—OH where m=1, 2 or 3;
Y represents —H, —CH₃, H₃C—(CH₂)ₖ— where k=1, 2, 3, 4, 5, 6, 7 or 8, —OH, —(CH₂)ₚ—OH where p=1, 2, 3 or 4, or —CHO.

Aldehydes that are preferably to be used as contemplated herein are described on pages 36 to 38 of the priority document as numbers 1 to 176.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of at least one alpha-substituted aldehyde from the group
2-hydroxypropanal (X=—OH, Y=—CH₃):

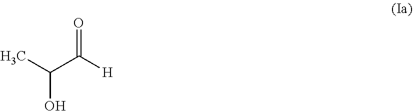

2-hydroxyhexanal (X=—OH, Y=H₃C—(CH₂)ₖ— where k=3):

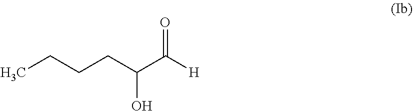

2-hydroxyoctanal (X=—OH, Y=H₃C—(CH₂)ₖ— where k=5):

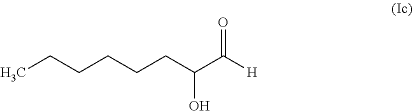

bromomalonaldehyde (X=Br, Y=—CH═):

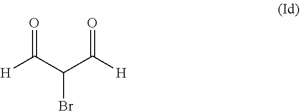

2-(2-hydroxyethoxy)acetaldehyde (X=—O—(CH₂)ₚ—OH where p=2, Y=—H):

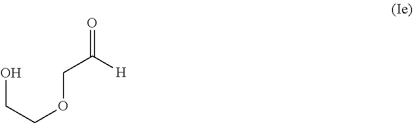

glyceraldehyde (X=—OH, Y=—(CH₂)ₚ—OH where p=1):

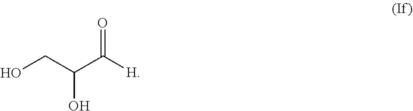

It has been found that succinimidyl esters can further enhance the effect of the agents as contemplated herein. In particular, significant increases in performance are observed with regard to the inner structural strengthening of keratin fibers and the prevention of washout.

Hair treatment agents which are preferred as contemplated herein additionally contain, based on the weight of the agent, from about 0.001 to about 10 wt. % of at least one succinimidyl ester.

Succinimidyl esters are esters of carboxylic acids having (optionally substituted) N-hydroxysuccinimide (NHS, IUPAC 1-hydroxy-2,5-pyrrolidinedione) and are also referred to as NHS esters. Hair treatment agents which are preferred as contemplated herein additionally contain, based on the weight of the agent, from about 0.01 to about 10 wt. %, preferably from about 0.05 to about 7.5 wt. %, more preferably from about 0.1 to about 6 wt. %, and in particular from about 0.15 to about 5 wt. %, of (a) succinimidyl ester(s) of formula (III)

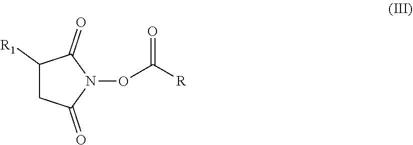

in which
R1 represents —H or an ionic group,
R represents an optionally substituted saturated or unsaturated, linear, branched or cyclic, aliphatic or aromatic hydrocarbon functional group having at least 5 C atoms.

Suitable ionic groups R1 include both anionic groups such as phosphate, phosphonate, phosphinate, sulfate, sulfonate, sulfinate, sulfenate, oxysulfonate, carboxylate groups and cationic groups such as substituted or unsubstituted ammonium groups. Zwitterionic/betainic groups such as carboxybetaine or sulfobetaine groups are also possible.

In succinimidyl esters which are preferred as contemplated herein, R1 represents —H, 'OSO₃⁻, —SO₃⁻, —SO₂⁻, —COO⁻, —NH₃⁺, —N(CH₃)H₂⁺, —N(CH₃)₂H⁺, —N(CH₃)₃⁺, —N⁺(CH₃)₂(CH₂)₂—COO⁻, or —N⁺(CH₃)₂(CH₂)₃—SO₃⁻.

Succinimidyl esters that are preferably to be used as contemplated herein are described on pages 41 to 43 of the priority document as numbers 1 to 72.

Very particularly preferred hair treatment agents as contemplated herein contain, based on their weight, from about 0.01 to about 10 wt. %, preferably from about 0.05 to about 7.5 wt. %, more preferably from about 0.1 to about 6 wt. %, and in particular from about 0.15 to about 5 wt. %, of at least one succinimidyl ester from the group

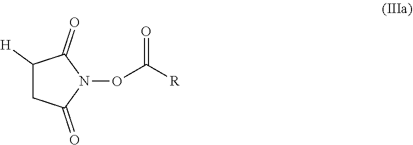

in which R represents -Ph or —(CH₂)nCH₃, where n=4, 5, 6, 7, 8, 9 or 10,

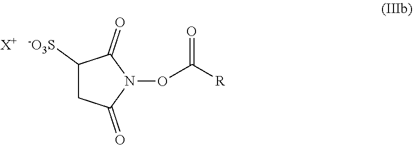

in which R represents -Ph or —(CH2)nCH₃, where n=4, 5, 6, 7, 8, 9 or 10 and
X represents H, a monovalent cation or the n-th part of an n-valent cation,

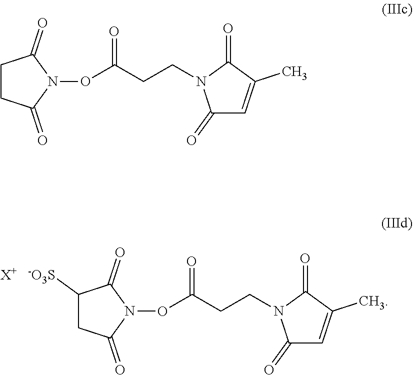

Complexing agents can further increase the effect of the agents as contemplated herein, complexing agents which are derived from polycarboxylic acids having been found to be particularly suitable.

Hair treatment agents which are preferred as contemplated herein contain, based on their weight, from about 0.001 to about 20 wt. % of complexing agents from the group tetrasodium-N,N-bis(carboxylatomethyl)-L-glutamate (tetrasodium glutamate diacetate, GLDA), pentasodium diethylenetriaminepentaacetate (DTPA), tetrasodium iminodisuccinate (IDS), tetrasodium ethylenediaminetetraacetate (EDTA), tetrasodium ethylenediamine disuccinic acid (EDDS), or trisodium hydroxyethyl ethylenediaminetriaccetic acid (HEDTA).

The use of the three complexing agents mentioned first is very particularly preferred. Extremely preferred hair treatment agents as contemplated herein contain, based on their weight, from about 0.001 to about 20 wt. %, preferably from about 0.005 to about 15 wt. %, more preferably from about 0.01 to about 10 wt. %, even more preferably from about 0.05 to about 7.5 wt. %, and in particular from about 0.1 to about 5 wt. %, of complexing agents from the group
a. tetrasodium-N,N-bis(carboxylatomethyl)-L-glutamate (tetrasodium glutamate diacetate, GLDA),
b. pentasodium diethylenetriaminepentaacetate (DTPA),
c. tetrasodium iminodisuccinate (IDS).

The agents as contemplated herein may contain only one of the three complexing agents mentioned. However, it is also possible for the agents as contemplated herein to contain two or all three of the above-mentioned complexing agents, the amount of all the complexing agents from the above-mentioned group contained in the agents being within an amount range of from about 0.01 to about 20 wt. %.

Hair treatment agents which are preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, of tetrasodium-N,N-bis(carboxylatomethyl)-L-glutamate (tetrasodium glutamate diacetate, GLDA).

Hair treatment agents which are also preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, of pentasodium diethylenetriaminepentaacetate (DTPA).

Hair treatment agents which are also preferred as contemplated herein contain, based on their weight, from about 0.005 to about 15 wt. %, preferably from about 0.01 to about 10 wt. %, more preferably from about 0.05 to about 7.5 wt. %, and in particular from about 0.1 to about 5 wt. %, of tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, of tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. %, of pentasodium diethylenetriaminepentaacetate (DTPA).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, of tetrasodium-N,N-bis(carboxylatomethyl)-L-glutamate (tetrasodium glutamate diacetate, GLDA), and from about 0.01 to about 10 wt. %, more preferably from about 0.05 to about 7.5 wt. %, and in particular from about 0.1 to about 5 wt. %, of tetrasodium iminodisuccinate (IDS).

Hair treatment agents which are also preferred as contemplated herein contain, based on their weight, from about 0.01 to about 10 wt. %, more preferably from about 0.05 to about 7.5 wt. %, and in particular from about 0.1 to about 5 wt. %, of pentasodium diethylenetriaminepentaacetate (DTPA), and from about 0.01 to about 10 wt. %, more preferably from about 0.05 to about 7.5 wt. %, and in particular from about 0.1 to about 5 wt. %, of tetrasodium iminodisuccinate (IDS).

Hair treatment agents which are also preferred as contemplated herein contain, based on their weight, from about 0.1 to about 5 wt. % of tetrasodium-N,N-bis(carboxylatomethyl)-L-glutamate (tetrasodium glutamate diacetate, GLDA), from about 0.1 to about 5 wt. % of pentasodium diethylenetriaminepentaacetate (DTPA), and from about 0.1 to about 5 wt. % of tetrasodium iminodisuccinate (IDS).

The hair treatment agents contain the above-described active ingredients preferably in a cosmetically acceptable carrier. Within the context of the present disclosure, this is understood to preferably mean an aqueous or aqueous-alcoholic carrier.

The cosmetic carrier preferably contains at least about 50 wt. %, more preferably at least about 60 wt. %, particularly preferably at least about 70 wt. %, and more particularly preferably at least about 75 wt. %, of water.

Furthermore, the cosmetic carrier may contain from about 0.01 to about 40 wt. %, preferably from about 0.05 to about 30 wt. %, and in particular from about 0.1 to about 20 wt. %, of at least one alcohol.

Suitable alcohols are, for example, ethanol, ethyl diglycol, 1-propanol, 2-propanol, isopropanol, 1,2-propylene glycol, glycerol, diglycerol, triglycerol, 1-butanol, 2-butanol, 1,2-butanediol, 1,3-butanediol, 1-pentanol, 2-pentanol, 1,2-pentanediol, 1,5-pentanediol, 1-hexanol, 2-hexanol, 1,2-hexanediol, 1,6-hexanediol, polyethylene glycols, sorbitol, sorbitan, benzyl alcohol, or mixtures of said alcohols.

Water-soluble alcohols are particularly preferred. Ethanol, 1,2-propylene glycol, glycerol, benzyl alcohol, and mixtures of said alcohols are particularly preferred.

It is advantageous for very good (scalp) skin compatibility of the hair treatment agents as contemplated herein if said agents have a slightly acidic pH.

It has been found that the agents as contemplated herein have particularly good skin compatibility and mildness in a pH range of from about 4.2 to about 5.8.

In a first preferred embodiment, the hair treatment agents as contemplated herein therefore preferably have a pH in the range of from about 4.2 to about 5.8, more preferably from about 4.3 to about 5.6, particularly preferably from about 4.4 to about 5.5, extremely preferably from about 4.5 to about 5.4, and particularly preferably from about 4.7 to about 5.3.

The hair treatment agents as contemplated herein may contain vegetable oils, vegetable butters, and/or vegetable waxes. These vegetable oil components give the hair improved combability and stylability and increase hair shine.

Carnauba wax, beeswax, and/or candelilla wax can preferably be used as suitable natural or vegetable waxes. Particularly preferred vegetable oil components are (sweet) almond oil, peach kernel oil, apricot kernel oil, amaranth seed oil, argan oil, olive oil, jojoba oil, cocoa butter, and/or shea butter.

The weight proportion of the at least one vegetable oil, vegetable butter, and/or vegetable wax in terms of the total weight of the hair treatment agents as contemplated herein is preferably from about 0.02 to about 2.50 wt. %, more preferably from about 0.03 to about 2.00 wt. %, particularly preferably from about 0.04 to about 1.50 wt. %, and in particular from about 0.05 to about 1.00 wt. %.

In addition to the aforementioned essential and optional components, the hair treatment agents as contemplated herein may contain, in a further preferred embodiment for further increasing the nourishing properties of the agents, at least one further active hair-conditioning ingredient, which can be selected from the group of
protein hydrolyzates,
vitamins,
plant extracts, and/or
glycerol.

Vitamins, provitamins, and vitamin precursors from the groups A, B, E, and H are particularly preferred. Particularly preferred are nicotinamide, biotin, pantolactone, and/or panthenol.

The weight proportion of the vitamin(s), vitamin derivative(s), and/or vitamin precursor(s) in terms of the total weight of the hair treatment agents is preferably from about 0.001 to about 2 wt. %, particularly preferably from about 0.005 to about 1 wt. %, and in particular from about 0.01 to about 0.5 wt. %.

It has been found that the hair treatment agents as contemplated herein are also suitable for use as an anti-dandruff preparation.

The total weight of anti-dandruff agents in terms of the total weight of the hair treatment agents can preferably be from about 0.01 to about 10 wt. %, more preferably from about 0.025 to about 7.5 wt. %, particularly preferably from about 0.05 to about 5 wt. %, and in particular from about 0.075 to about 3 wt. %.

Suitable anti-dandruff active ingredients can be selected from piroctone olamine, climbazole, zinc pyrithione, ketoconazole, salicylic acid, sulfur, selenium sulfide, tar preparations, undecenoic acid derivatives, burdock root extracts, poplar extracts, stinging nettle extracts, walnut shell extracts, birch extracts, willow bark extracts, rosemary extracts, and/or arnica extracts.

Climbazole, zinc pyrithione, and piroctone olamine are preferred.

The agents as contemplated herein can be formulated as what are referred to as rinse-off products, i.e. are rinsed out of the hair again after a specific contact time. This contact time is preferably less than an hour, i.e. the consumer preferably does not leave the products in the hair until the next time the hair is washed.

A further subject of the present disclosure is therefore a method for treating hair in which an agent as contemplated herein is applied to dry or wet hair, is left there for a period of from about 10 to about 300 seconds, and is rinsed out thereafter.

The agents as contemplated herein can also be formulated as what are referred to as leave-on products, i.e. are not rinsed out of the hair, but are instead left there until the next time the hair is washed.

A further subject of the present disclosure is therefore a method for treating hair in which an agent as contemplated herein is applied to dry or wet hair and is left there until the next time the hair is washed.

The agents as contemplated herein lead to significantly increased strengthening of the internal and external hair structure. A further subject of the present disclosure is therefore the use of agents as contemplated herein for strengthening the hair structure, in particular the internal hair structure.

Within the meaning of the present disclosure, “structure strengthening” is understood to mean a reduction in the damage to keratin fibers caused by a wide range of influences. The re-establishment of the natural strength, for example, plays a key role in this respect.

Restructured fibers are distinguished for example by improved shine, by an improved feel, and by higher combability. In addition, they have optimized strength and elasticity. Successful structure strengthening or restructuring can be verified physically as an increase in the melting point by comparison with damaged fibers.

The agents as contemplated herein also lead to a considerably increased stability of artificial colors against the washout of the color. Chemically colored hair can thus be washed much more frequently using the agents as contemplated herein, without this leading to undesired bleeding or fading of the color.

A further subject of the present disclosure is therefore the use of agents as contemplated herein for reducing the washout of color from chemically colored hair.

The statements made about the agents as contemplated herein apply, mutatis mutandis, to particularly preferred embodiments of the method as contemplated herein and to the uses as contemplated herein.

### EXAMPLES

All values in wt. %.

### Hair Shampoo:

| | 1 | 2 | 3 | 4 | 5 | 6 |
|---|---|---|---|---|---|---|
| Sodium laureth sulfate | 10 | 10 | 10 | 10 | 10 | 10 |
| Cocoamidopropyl betaine | 4.0 | 4.0 | 4.0 | 4.0 | 4.0 | 4.0 |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.8 | 0.8 | 0.8 | 0.8 | 0.8 | 0.8 |
| Monoethanol ammonium | 0.1 | 0.5 | 0.2 | 0.1 | 0.2 | 0.15 |
| hexanoate | | | | | | |
| Panthenol | 0.5 | 0.5 | 0.5 | 0.5 | 0.5 | 0.5 |
| 2-hydroxyoctanal | 1.0 | 1.0 | 1.0 | 1.0 | 1.0 | 1.0 |
| Amodimethicone/morpholinomethyl | 1.0 | 2.0 | 3.5 | 1.0 | 0.8 | 1.0 |
| silsesquioxane copolymer | | | | | | |
| Laureth-2 | 1.2 | 1.2 | 1.2 | 1.2 | 1.2 | 1.2 |
| Sodium chloride | 1.3 | 1.0 | 1.0 | 1.2 | 1.1 | 1.3 |
| Magnesium chloride | 1.5 | 1.2 | 2.0 | 1.5 | 1.5 | 1.5 |
| Water, preservative, perfume oils | up to 100 | up to 100 | up to 100 | up to 100 | up to 100 | up to 100 |
| | 7 | 8 | 9 | 10 | 11 | 12 |
| Sodium laureth sulfate | 10 | 10 | 10 | 10 | 10 | 10 |
| Cocoamidopropyl betaine | 4.0 | 4.0 | 4.0 | 4.0 | 4.0 | 4.0 |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.8 | 0.8 | 0.8 | 0.8 | 0.8 | 0.8 |
| Monoethanol ammonium | 0.1 | 0.5 | 0.2 | 0.1 | 0.2 | 0.15 |
| hexanoate | | | | | | |
| Glyceraldehyde | 0.10 | — | — | — | — | — |
| 2-hydroxypropanal | — | 0.12 | — | — | — | — |
| 2-hydroxyhexanal | — | — | 0.15 | — | — | — |
| 2-hydroxyoctanal | — | — | — | 0.10 | — | — |
| 2-(2-hydroxyethoxy)acetaldehyde | — | — | — | — | 0.12 | — |
| Bromomalonaldehyde | — | — | — | — | — | 0.10 |
| Panthenol | 0.5 | 0.5 | 0.5 | 0.5 | 0.5 | 0.5 |
| 2-hydroxyoctanal | 1.0 | 1.0 | 1.0 | 1.0 | 1.0 | 1.0 |
| Amodimethicone/morpholinomethyl | 1.0 | 2.0 | 3.5 | 1.0 | 0.8 | 1.0 |
| silsesquioxane copolymer | | | | | | |
| Laureth-2 | 1.2 | 1.2 | 1.2 | 1.2 | 1.2 | 1.2 |
| Sodium chloride | 1.3 | 1.0 | 1.0 | 1.2 | 1.1 | 1.3 |
| Calcium chloride | 1.5 | 1.2 | 2.0 | 1.5 | 1.5 | 1.5 |
| Water, preservative, perfume oils | up to 100 | up to 100 | up to 100 | up to 100 | up to 100 | up to 100 |

### Hair Conditioner:

| | 1 | 2 | 3 | 4 | 5 | 6 |
|---|---|---|---|---|---|---|
| Quaternium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Cetearyl alcohol | 5.0 | 5.0 | 5.0 | 5.0 | 5.0 | 5.0 |
| Guar hydroxypropyltrimonium | 0.2 | 0.2 | 0.2 | 0.2 | 0.2 | 0.2 |
| chloride | | | | | | |
| Polyquaternium-37 | 0.4 | 0.4 | 0.4 | 0.4 | 0.4 | 0.4 |
| Hydrolyzed keratin | 0.1 | 0.1 | 0.1 | 0.1 | 0.1 | 0.1 |
| Shea butter | 1.5 | 1.5 | 1.5 | 1.5 | 1.5 | 1.5 |
| Behenoyl PG-trimoniumchloride | 1.5 | 1.5 | 1.5 | 1.5 | 1.5 | 1.5 |
| Distearoylethyl | 0.3 | 0.3 | 0.3 | 0.3 | 0.3 | 0.3 |
| hydroxyethylmonium | | | | | | |
| methosulfate | | | | | | |
| Behentrimonium chloride | 0.4 | 0.4 | 0.4 | 0.4 | 0.4 | 0.4 |
| Lactic acid | 0.1 | 0.1 | 0.1 | 0.1 | 0.1 | 0.1 |
| Trimethyl ammonium octanoate | 0.2 | 0.5 | 1.0 | 1.2 | 0.8 | 0.6 |
| Amodimethicone | 0.8 | 0.8 | 0.8 | 0.8 | 0.8 | 0.8 |
| Calcium chloride | 1.5 | 1.2 | 2.0 | 1.5 | 1.5 | 1.5 |
| Water, preservative, perfume oils | up to 100 | up to 100 | up to 100 | up to 100 | up to 100 | up to 100 |
| | 7 | 8 | 9 | 10 | 11 | 12 |
| Quaternium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Cetearyl alcohol | 5.0 | 5.0 | 5.0 | 5.0 | 5.0 | 5.0 |
| Guar | 0.2 | 0.2 | 0.2 | 0.2 | 0.2 | 0.2 |
| hydroxypropyltrimonium | | | | | | |
| chloride | | | | | | |
| Polyquaternium-37 | 0.4 | 0.4 | 0.4 | 0.4 | 0.4 | 0.4 |
| Hydrolyzed keratin | 0.1 | 0.1 | 0.1 | 0.1 | 0.1 | 0.1 |
| Shea butter | 1.5 | 1.5 | 1.5 | 1.5 | 1.5 | 1.5 |
| Behenoyl PG-trimoniumchloride | 1.5 | 1.5 | 1.5 | 1.5 | 1.5 | 1.5 |
| Distearoylethyl | 0.3 | 0.3 | 0.3 | 0.3 | 0.3 | 0.3 |
| hydroxyethylmonium | | | | | | |
| methosulfate | | | | | | |
| Behentrimonium chloride | 0.4 | 0.4 | 0.4 | 0.4 | 0.4 | 0.4 |
| Lactic acid | 0.1 | 0.1 | 0.1 | 0.1 | 0.1 | 0.1 |
| Trimethyl ammonium octanoate | 0.2 | 0.5 | 1.0 | 1.2 | 0.8 | 0.6 |
| Glyceraldehyde | 1.2 | — | — | — | — | — |
| 2-hydroxypropanal | — | 1.0 | — | — | — | — |
| 2-hydroxyhexanal | — | — | 2.0 | — | — | — |
| 2-hydroxyoctanal | — | — | — | 1.5 | — | — |
| 2-(2-hydroxyethoxy)acetaldehyde | — | — | — | — | 1.2 | — |
| Bromomalonaldehyde | — | — | — | — | — | 1.5 |
| Amodimethicone | 0.8 | 0.8 | 0.8 | 0.8 | 0.8 | 0.8 |
| Calcium chloride | 1.5 | 1.2 | 2.0 | 1.5 | 1.5 | 1.5 |
| Water, preservative, perfume oils | up to 100 | up to 100 | up to 100 | up to 100 | up to 100 | up to 100 |

### Succinimidyl Esters Used:

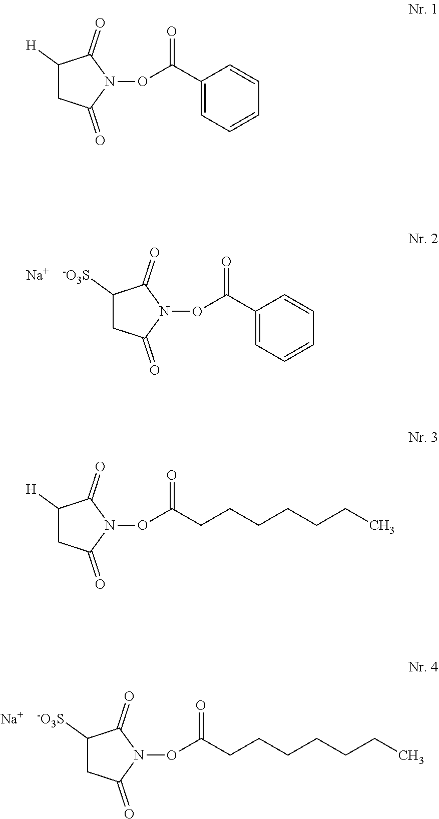

### Hair Shampoo:

| | 13 | 14 | 15 | 16 | 17 | 18 |
|---|---|---|---|---|---|---|
| Sodium laureth sulfate | 10 | 10 | 10 | 10 | 10 | 10 |
| Cocoamidopropyl betaine | 4.0 | 4.0 | 4.0 | 4.0 | 4.0 | 4.0 |
| Ammonium lauryl sulfate | 4.0 | 4.0 | 4.0 | 4.0 | 4.0 | 4.0 |
| Polyquaternium-10 | 0.3 | 0.3 | 0.3 | 0.3 | 0.3 | 0.3 |
| Cocamide MEA | 0.8 | 0.8 | 0.8 | 0.8 | 0.8 | 0.8 |
| PEG-7 glyceryl cocoate | 1.0 | 1.0 | 1.0 | 1.0 | 1.0 | 1.0 |
| Nicotinamide | 0.3 | 0.3 | 0.3 | 0.3 | 0.3 | 0.3 |
| Citric acid | 0.8 | 0.8 | 0.8 | 0.8 | 0.8 | 0.8 |
| Succinimidyl ester no. 1 | 0.1 | — | — | — | 0.1 | — |
| Succinimidyl ester no. 2 | — | 0.15 | — | — | — | — |
| Succinimidyl ester no. 3 | — | — | 0.1 | — | — | 0.1 |
| Succinimidyl ester no. 4 | — | — | — | 0.15 | — | — |
| Glyceraldehyde | — | — | — | — | 0.1 | — |
| Bromomalonaldehyde | — | — | — | — | — | 0.2 |
| Panthenol | 0.5 | 0.5 | 0.5 | 0.5 | 0.5 | 0.5 |
| 2-hydroxyoctanal | 1.0 | 1.0 | 1.0 | 1.0 | 1.0 | 1.0 |
| Trimethyl ammonium octanoate | 0.2 | 0.5 | 1.0 | 1.2 | 0.8 | 0.6 |
| Amodimethicone/morpholinomethyl | 1.0 | 2.0 | 3.5 | 1.0 | 0.8 | 1.0 |
| silsesquioxane copolymer | | | | | | |
| Laureth-2 | 1.2 | 1.2 | 1.2 | 1.2 | 1.2 | 1.2 |
| Sodium chloride | 1.3 | 1.0 | 1.0 | 1.2 | 1.1 | 1.3 |
| Magnesium chloride | 1.5 | 1.2 | 2.0 | 1.5 | 1.5 | 1.5 |
| Water, preservative, perfume oils | up to 100 | up to 100 | up to 100 | up to 100 | up to 100 | up to 100 |

### Hair Conditioner:

| | 13 | 14 | 15 | 16 | 17 | 18 |
|---|---|---|---|---|---|---|
| Quaternium-87 | 0.75 | 0.75 | 0.75 | 0.75 | 0.75 | 0.75 |
| Glycol distearate | 1.0 | 1.0 | 1.0 | 1.0 | 1.0 | 1.0 |
| Cetearyl alcohol | 5.0 | 5.0 | 5.0 | 5.0 | 5.0 | 5.0 |
| Guar hydroxypropyltrimonium | 0.2 | 0.2 | 0.2 | 0.2 | 0.2 | 0.2 |
| chloride | | | | | | |
| Polyquaternium-37 | 0.4 | 0.4 | 0.4 | 0.4 | 0.4 | 0.4 |
| Hydrolyzed keratin | 0.1 | 0.1 | 0.1 | 0.1 | 0.1 | 0.1 |
| Shea butter | 1.5 | 1.5 | 1.5 | 1.5 | 1.5 | 1.5 |
| Behenoyl PG-trimoniumchloride | 1.5 | 1.5 | 1.5 | 1.5 | 1.5 | 1.5 |
| Distearoylethyl | 0.3 | 0.3 | 0.3 | 0.3 | 0.3 | 0.3 |
| hydroxyethylmonium | | | | | | |
| methosulfate | | | | | | |
| Behentrimonium chloride | 0.4 | 0.4 | 0.4 | 0.4 | 0.4 | 0.4 |
| Monoethanol ammonium | 0.1 | 0.5 | 0.2 | 0.1 | 0.2 | 0.15 |
| hexanoate | | | | | | |
| Lactic acid | 0.1 | 0.1 | 0.1 | 0.1 | 0.1 | 0.1 |
| Succinimidyl ester no. 1 | 0.1 | — | — | — | — | — |
| Succinimidyl ester no. 2 | — | 0.15 | — | — | 0.1 | — |
| Succinimidyl ester no. 3 | — | — | 0.1 | — | — | — |
| Succinimidyl ester no. 4 | — | — | — | 0.15 | — | 0.1 |
| 2-hydroxyhexanal | -— | — | — | — | 0.1 | — |
| 2-hydroxyoctanal | — | — | — | — | — | 0.2 |
| Amodimethicone | 0.8 | 0.8 | 0.8 | 0.8 | 0.8 | 0.8 |
| Calcium chloride | 1.5 | 1.2 | 2.0 | 1.5 | 1.5 | 1.5 |
| Water, preservative, perfume oils | up to 100 | up to 100 | up to 100 | up to 100 | up to 100 | up to 100 |

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A hair treatment agent comprising, based on its weight,
a) From about 0.001 to about 10 wt. % of at least one amine salt of a carboxylic acid;
b) at least one bivalent or trivalent metal salt;
c) at least one organic acid,
with the proviso that the agent comprises, based on its weight, from about 0.01 to about 10 wt. % of at least one amine salt from the group 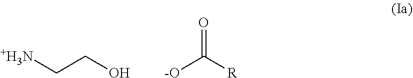
in which R represents -Ph or —(CH₂)nCH3, where n=4, 5, 6, 7, 8, 9 or 10, 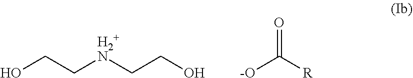
in which R represents -Ph or —(CH₂)nCH3, where n=4, 5, 6, 7, 8, 9 or 10, 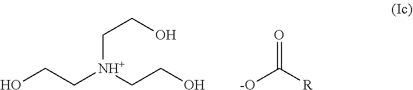
in which R represents -Ph or —(CH₂)nCH₃, where n=4, 5, 6, 7, 8, 9 or 10, 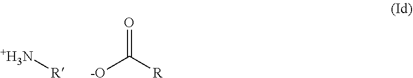
in which R and R′ represent, independently of one another, -Ph or —(CH₂)nCH3, where n=4, 5, 6, 7, 8, 9 or 10.

2. The hair treatment agent according to claim 1, wherein it comprises, based on its weight, from about 0.01 to about 10 wt. %, of amine salt(s) of carboxylic acid(s) of formula (I) 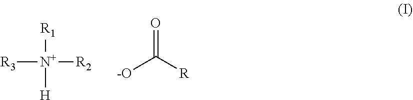
in which
R1, R2 and R3 are selected, independently of one another, from —H, —CH₃, —CH₂CH₃, —CH₂CH₂OH,
R represents an optionally substituted saturated or unsaturated, linear, branched or cyclic, aliphatic or aromatic hydrocarbon functional group having at least 2 C atoms.

3. The hair treatment agent according to claim 1, wherein it comprises, based on its weight, from about 0.05 to about 7.5 wt. %, of at least one amine salt from the group 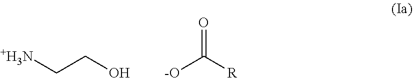
in which R represents -Ph or —(CH₂)nCH3, where n=4, 5, 6, 7, 8, 9 or 10, 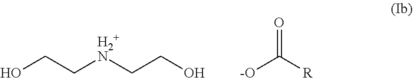
in which R represents -Ph or —(CH₂)nCH3, where n=4, 5, 6, 7, 8, 9 or 10, 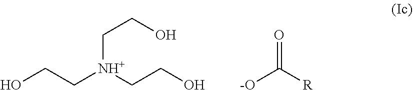
in which R represents -Ph or —(CH₂)nCH₃, where n=4, 5, 6, 7, 8, 9 or 10, 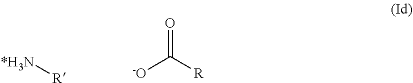
in which R and R′ represent, independently of one another, -Ph or —(CH₂)nCH3, where n=4, 5, 6, 7, 8, 9 or 10.

4. The hair treatment agent according to claim 1, wherein it comprises, based on its weight, from about 0.01 to about 10 wt. %, of at least one bivalent or trivalent metal salt from the group of organic or inorganic copper, zinc, iron(II), calcium, magnesium, iron(II), lanthanum(III), and/or aluminum salts.

5. The hair treatment agent according to claim 1, wherein it comprises, based on its weight, from about 0.001 to about 10 wt. %, of organic acid(s) from the group of formic acid, oxalic acid, maleic acid, lactic acid, tartaric acid, succinic acid, or citric acid.

6. The hair treatment agent according to claim 1, wherein it comprises, based on its weight, from about 0.3 to about 20 wt. %, of amphoteric surfactant(s).

7. The hair treatment agent according to claim 1, wherein it comprises, based on the weight of the agent, from about 0.01 to about 3 wt. %, of at least one polymer from the group of cationic cellulose polymers and/or cationic guar derivatives.

8. The hair treatment agent according to claim 1, wherein it comprises, based on the weight of the agent, from about 0.05 to about 20 wt. %, of behenyl trimethyl ammonium chloride.

9. The hair treatment agent according to claim 1, wherein it has a pH of from about 2.0 to about 7.0.

10. The hair treatment agent according to claim 1, wherein it additionally comprises, based on the weight of the agent, from about 0.001 to about 20 wt. % of at least one alpha-substituted aldehyde.

11. The hair treatment agent according to claim 1, wherein it additionally comprises, based on the weight of the agent, from about 0.01 to about 10 wt. % of alpha-substituted aldehyde(s) of formula (III)
Y—CH(X)—CHO (III)
in which
X represents —OH, —Cl, —Br, —I, —O—(CH₂)ₙ where n=1, 2, 3 or 4, or —O—(CH₂)ₘ—OH where m=1, 2 or 3;
Y represents —H, —CH₃, H₃C—(CH₂)ₖ— where k=1, 2, 3, 4, 5, 6, 7 or 8, —OH, —(CH₂)ₚ—OH where p=1, 2, 3 or 4, or —CHO.

12. The hair treatment agent according to claim 1, wherein it comprises, based on its weight, from about 0.01 to about 10 wt. %, of at least one alpha-substituted aldehyde from the group
2-hydroxypropanal (X=—OH, Y=—CH₃): 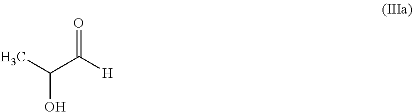
2-hydroxyhexanal (X=—OH, Y=H₃C—(CH₂)ₖ— where k=3): 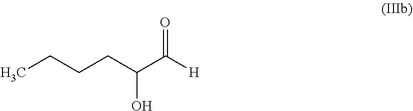
2-hydroxyoctanal (X=—OH, Y=H₃C—(CH₂)ₖ— where k=5): 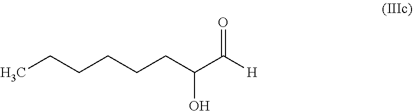
bromomalonaldehyde (X=Br, Y=—CH═): 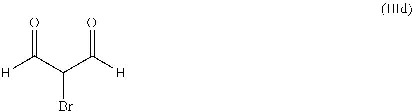
2-(2-hydroxyethoxy)acetaldehyde (X=—O—(CH₂)ₚ—OH where p=2, Y=—H): 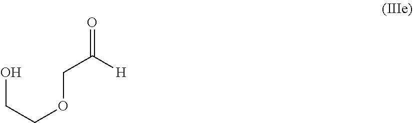
glyceraldehyde (X=—OH, Y=—(CH₂)ₚ—OH where p=1):

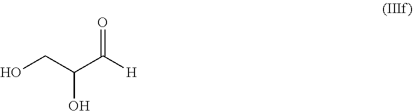

13. The hair treatment agent according to claim 1, wherein it additionally comprises, based on the weight of the agent, from about 0.001 to about 10 wt. % of at least one succinimidyl ester.

14. The hair treatment agent according to claim 1, wherein it additionally comprises, based on the weight of the agent, from about 0.01 to about 10 wt. %, of (a) succinimidyl ester(s) of formula (II) 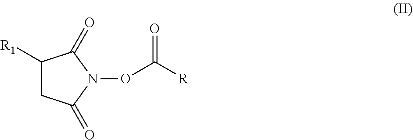
in which
R1 represents —H or an ionic group,
R represents an optionally substituted saturated or unsaturated, linear, branched or cyclic, aliphatic or aromatic hydrocarbon functional group having at least 5 C atoms.

15. The hair treatment agent according to claim 1, wherein it comprises, based on its weight, from about 0.01 to about 10 wt. %, of at least one succinimidyl ester from the group 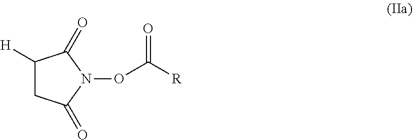
in which R represents -Ph or —(CH₂)nCH3, where n=4, 5, 6, 7, 8, 9 or 10, 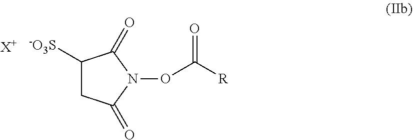
in which R represents -Ph or —(CH2)nCH3, where n=4, 5, 6, 7, 8, 9 or 10 and
X represents H, a monovalent cation or the n-th part of an n-valent cation,

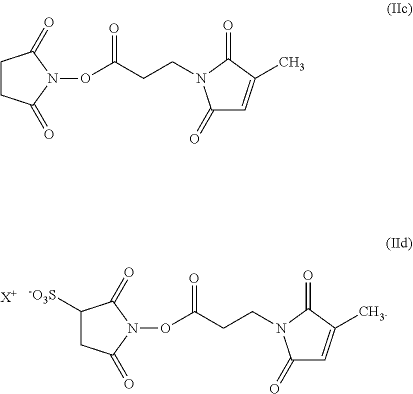

16. The hair treatment agent according to claim 1, wherein it comprises, based on its weight, from about 0.001 to about 20 wt. % of complexing agents from the group tetrasodium-N,N-bis(carboxylatomethyl)-L-glutamate (tetrasodium glutamate diacetate, GLDA), pentasodium diethylenetriaminepentaacetate (DTPA), tetrasodium iminodisuccinate (IDS), tetrasodium ethylenediaminetetraacetate (EDTA), tetrasodium ethylenediamine disuccinic acid (EDDS), or trisodium hydroxyethyl ethylenediaminetriaccetic acid (HEDTA).

17. The hair treatment agent according to claim 1, wherein it comprises, based on its weight, from about 0.001 to about 20 wt. %, of complexing agents from the group
a. tetrasodium-N,N-bis(carboxylatomethyl)-L-glutamate (tetrasodium glutamate diacetate, GLDA) 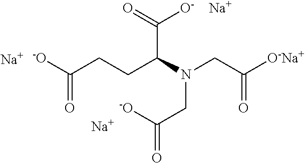
b. pentasodium diethylenetriaminepentaacetate (DTPA) 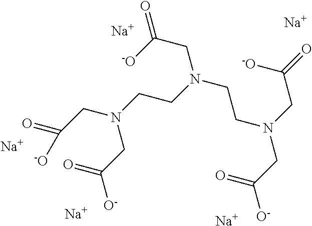
c. tetrasodium iminodisuccinate (IDS)

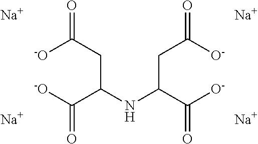

18. A method for treating hair, wherein an agent according to claim 1 is applied to dry or wet hair, is left there for a period of from about 10 to about 300 seconds, and is rinsed out thereafter.

19. A method for treating hair, wherein an agent according to claim 1 is applied to dry or wet hair and is left there until the next time the hair is washed.

20. (canceled)

21. The hair treatment agent according to claim 1,
wherein it comprises, based on its weight, from about 0.15 to about 5 wt. %, of at least one amine salt from the group 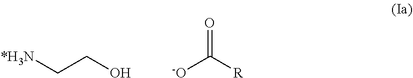
in which R represents -Ph or —(CH₂)nCH3, where n=4, 5, 6, 7, 8, 9 or 10, 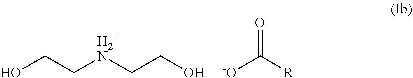
in which R represents -Ph or —(CH₂)nCH3, where n=4, 5, 6, 7, 8, 9 or 10, 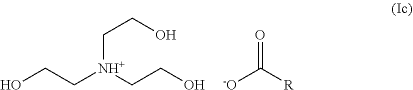
in which R represents -Ph or —(CH₂)nCH₃, where n=4, 5, 6, 7, 8, 9 or 10, 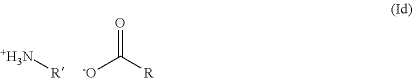
in which R and R′ represent, independently of one another, -Ph or —(CH₂)nCH3, where n=4, 5, 6, 7, 8, 9 or 10,
wherein it comprises, based on its weight, from about 0.3 to about 3 wt. %, of at least one bivalent or trivalent metal salt from the group of organic or inorganic copper, zinc, iron(II), calcium, magnesium, iron(II), lanthanum(III), and/or aluminum salts,
wherein it comprises, based on its weight, from about 0.1 to about 2 wt. %, of organic acid(s) from the group of formic acid, oxalic acid, maleic acid, lactic acid, tartaric acid, succinic acid, or citric acid,
wherein it comprises, based on its weight, from about 1 to about 5 wt. %, of amphoteric surfactant(s),
wherein it comprises, based on the weight of the agent, from about 0.15 to about 0.8 wt. %, of at least one polymer from the group of cationic cellulose polymers and/or cationic guar derivatives,
wherein it comprises, based on the weight of the agent, from about 0.5 to about 7 wt. %, of behenyl trimethyl ammonium chloride,
wherein it has a pH of from about 3.0 to about 5.0,
wherein it additionally comprises, based on the weight of the agent, from about 0.15 to about 5 wt. % of at least one alpha-substituted aldehyde from the group
2-hydroxypropanal (X=—OH, Y=—CH₃): 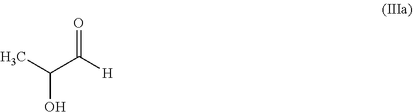
2-hydroxyhexanal (X=—OH, Y=H₃C—(CH₂)ₖ— where k=3): 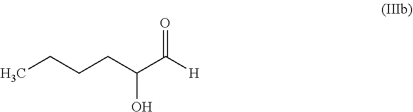
2-hydroxyoctanal (X=—OH, Y=H₃C—(CH₂)ₖ— where k=5): 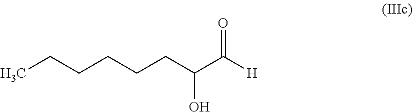
bromomalonaldehyde (X=Br, Y=—CH═): 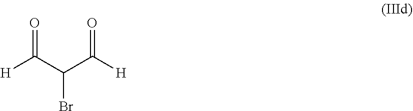
2-(2-hydroxyethoxy)acetaldehyde (X=—O—(CH₂)ₚ—OH where p=2, Y=—H): 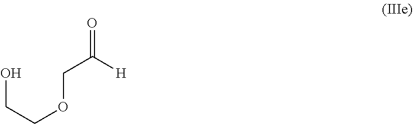
glyceraldehyde (X=—OH, Y=—(CH₂)ₚ—OH where p=1): 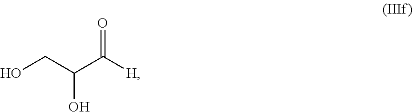
wherein it additionally comprises, based on the weight of the agent, from about 0.15 to about 5 wt. % of at least one succinimidyl ester from the group 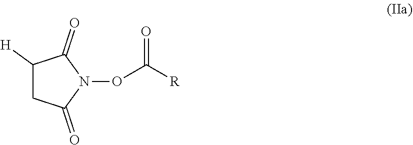
in which R represents -Ph or —(CH₂)nCH3, where n=4, 5, 6, 7, 8, 9 or 10, 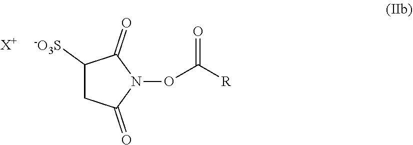
n which R represents -Ph or —(CH2)nCH3, where n=4, 5, 6, 7, 8, 9 or 10 and
X represents H, a monovalent cation or the n-th part of an n-valent cation, 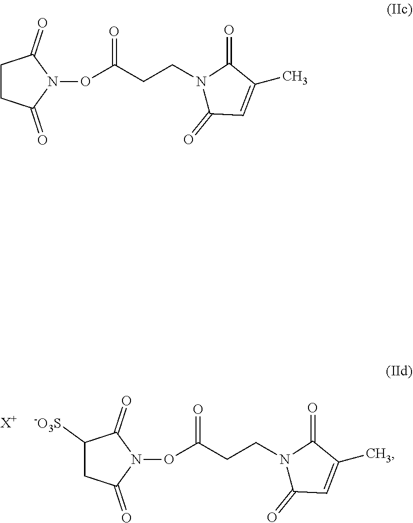
and
wherein it comprises, based on its weight, from about 0.1 to about 5 wt. %, of complexing agents from the group
a. tetrasodium-N,N-bis(carboxylatomethyl)-L-glutamate (tetrasodium glutamate diacetate, GLDA) 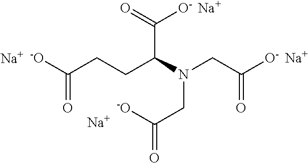
b. pentasodium diethylenetriaminepentaacetate (DTPA) 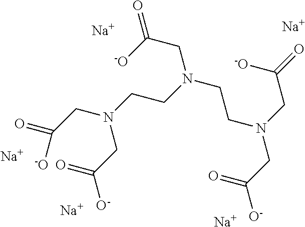
c. tetrasodium iminodisuccinate (IDS) [chemical structure]
